(19) United States
(12) Patent Application Publication
(10) Pub. No.: US 2009/0312559 A1
(43) Pub. Date: Dec. 17, 2009

(54) CATALYTIC ENANTIOSELECTIVE SILYLATIONS OF SUBSTRATES

(76) Inventors: Amir H. Hoveyda, Lincoln, MA (US); Marc L. Snapper, Ashland, MA (US); Jason Rodrigo, Brighton, MA (US); Yu Zhao, Brighton, MA (US)

Correspondence Address: EDWARDS ANGELL PALMER & DODGE LLP, P.O. BOX 55874, BOSTON, MA, 02205, US

(21) Appl. No.: 12/160,411

(22) PCT Filed: Jan. 10, 2007
(86) PCT No.: PCT/US07/00710

§ 371 Date: Jan. 16, 2009

Related U.S. Application Data
(60) Provisional application No. 60/757,856, filed on Jan. 10, 2006.

(51) Int. Cl. C07F 7/08; C07D 233/61

(52) U.S. Cl. 548/340.1; 556/436; 556/449

(57) ABSTRACT

The present invention provides methods, compositions and systems for silylation of substrates, including direct asymmetric silylation of a substrate to provide enantiomerically enriched silylated products.

### RELATED APPLICATIONS

This application claims the benefit of U.S. provisional patent application No. 60/757,856, filed Jan. 10, 2006, the contents of which are hereby incorporated by reference in their entirety.

### GOVERNMENT SUPPORT

The invention was supported, in whole or in part, by the National Institutes of Health under Contract/Grant No. R01-GM57212. The Government has certain rights in the invention.

### BACKGROUND OF THE INVENTION

Enzymatic transformations can generate useful chiral building blocks with high enantiopurity. Enzymatic acylation, for example, can proceed with high enantioselectivity in the desymmetrization of meso-diols, as well as provide useful selectivities in various kinetic resolutions. Likewise, non-enzymatic, catalytic asymmetric acylations have also proven useful for the production of chiral starting materials. While the substrate scope and selectivity in these transformations can be quite remarkable, the resulting acylated products often require additional manipulations to proceed in any particular synthetic sequence. This shortcoming can be attributed to the reactivity of the acyl group introduced in the asymmetric transformation. This base- and nucleophile-reactive moiety often needs to be replaced with a more useful and robust functionality.

One of the most common temporary functionalities tolerated in complex molecule synthesis to mask a hydroxyl group is the trialkyl-silyl moiety. The availability of well-established and selective methods for introducing and removing this functionality is in part responsible for its common use. However, there is a need in the art for a method capable of providing direct asymmetric silylation of a substrate. In addition, there is a need in the art for catalysts to be used in such a method.

### SUMMARY OF THE INVENTION

The present invention provides methods, compounds and systems for direct asymmetric silylation of a substrate to provide enantiomerically enriched silylated products. The presently disclosed methods, compounds and systems feature novel catalysts. A benefit of the invention is the ability to bypass the need to access chiral materials through acyl intermediates. This step-saving process will be of interest to those involved in the asymmetric synthesis of complex molecules.

Thus, in one aspect, the invention provides a method for direct asymmetric silylation of an alcohol. The method includes the step of reacting the alcohol with a silylating agent in the presence of a chiral catalyst, to produce an enantiomerically enriched silylated product, preferably in a single step (e.g., without isolation of intermediate compounds and the like).

In certain embodiments, the catalyst is represented by the formula:

in which:
R₁ is H, C₁-C₆ alkyl, aryl, or heteroaryl;
R₂ is, independently for each occurrence, C₁-C₆ alkyl, aryl, or heteroaryl;
Y is O, S or N—R₃;
Z is, independently for each occurrence, O or S;
R₃ is H, C₁-C₆ alkyl, aryl, or heteroaryl;
R₄ is (CH₂)ₘ—X;
X is aryl or heteroaryl;
m is 0, 1 or 2; and
n is an integer from 1 to 10;

or a salt thereof.

In certain embodiments, Y is N—R₃. In certain embodiments, Z is O for each occurrence. In certain embodiments, the catalyst is represented by the formula:

in which

R₁ is H, C₁-C₆ alkyl, aryl, or heteroaryl;

R₂ is C₁-C₆ alkyl, aryl, or heteroaryl;

R₃ is H, C₁-C₆ alkyl, aryl, or heteroaryl;

R₄ is (CH₂)ₘ—X;

X is aryl or heteroaryl;

m is 0, 1 or 2; and

n is an integer from 1 to 10;

or a salt thereof.

In certain embodiments, X is heteroaryl; or X is a nitrogen-containing 5- or 6-membered heteroaryl group; or X is a 1-methylimidazol-2-yl group.

In certain embodiments, the catalyst can be represented by the formula:

in which:

R₂ is a straight or branched alkyl group;

R₅ is an aryl or alkyl group;

R₆ is a lower alkyl;

R₇ is a lower alkyl; and

n=1, 2, 3, 4, 5, 6, 7, 8, 9, or 10;

or a salt thereof.

In certain embodiments, the catalyst can be represented by the formula:

wherein:

R₂ is a straight or branched alkyl group;

R₅ is:

R₆ is a lower alkyl; and

R₇ is a lower alkyl;

n=1, 2, 3, 4, 5, 6, 7, 8, 9, or 10.

In certain embodiments, R₅ is phenyl. In certain embodiments, R₅ is t-butyl. In certain embodiments, n=1. In certain embodiments, R₆ is a methyl group. In certain embodiments, R₇ is a methyl group. In certain embodiments, R₂ is:

In certain of the embodiments, the alcohol is a diol, such as a 1,2-diol (including a 1,2 cyclic diol) or a 1,3-diol (including a 1,3 cyclic diol).

In another aspect, the invention provides a compound or catalyst represented by the formula:

in which:

R₂ is a straight or branched alkyl group;

R₅ is an aryl or alkyl group;

R₆ is a lower alkyl;

R₇ is a lower alkyl; and

n=1, 2, 3, 4, 5, 6, 7, 8, 9, or 10;

or a salt thereof.

In certain embodiments, R₅ is phenyl; in certain embodiments, R₅ is t-butyl. In certain embodiments, n=1. In certain embodiments, R₇ is a methyl group. In certain embodiments, R₆ is a methyl group. In certain embodiments, R₃ is:

In another aspect, the invention provides a compound (or catalyst) represented by the formula:

in which R₅ is phenyl or t-butyl; and R₅ and R₉ are each independently H, alkyl or aryl;

or a salt thereof.

In certain embodiments, the compound is represented by the formula:

In certain embodiments, R₅ is t-butyl. In certain embodiments, R₅ is phenyl. In certain embodiments, R₈ and R₉ are each independently selected from methyl and ethyl. In certain embodiments, R₈ and R₉ are each methyl.

In another aspect, the invention provides a method of silylating a hydroxy group of a compound comprising at least one hydroxy group. The method includes the step of contacting the compound with a catalyst of the formula:

in which:
R₁ is H, C₁-C₆ alkyl, aryl, or heteroaryl;
R₂ is, independently for each occurrence, C₁-C₆ alkyl, aryl, or heteroaryl;
Y is O, S or N—R₃;
Z is, independently for each occurrence, O or S;
R₃ is H, C₁-C₆ alkyl, aryl, or heteroaryl;
R₄ is (CH₂)ₘ—X;
X is aryl or heteroaryl;
m is 0, 1 or 2; and
n is an integer from 1 to 10;

or a salt thereof;

in the presence of a silylating reagent and a base; under conditions such that a hydroxy group of the compound is silylated.

In certain embodiments, the compound is a diol, such as a 1,2-diol or a 1,3-diol. In certain embodiments, the compound is a triol. In certain embodiments, the compound is a beta-hydroxy ketone.

In certain embodiments, the base is an organic base.

In certain embodiments, the catalyst is represented by the formula:

wherein:

R₂ is a straight or branched alkyl group;

R₅ is an aryl or alkyl group;

R₆ is a lower alkyl;

R₇ is a lower alkyl; and

n=1, 2, 3, 4, 5, 6, 7, 8, 9, or 10;

or a salt thereof.

In certain embodiments, the catalyst is represented by the formula:

in which R₅ is phenyl or t-butyl; and R₈ and R₉ are each independently H, alkyl or aryl; or a salt thereof. In certain embodiments, R₈ and R₉ are each independently selected from methyl and ethyl. In certain embodiments, R₈ and R₉ are each methyl.

In another aspect, the invention provides a method of kinetic resolution of a mixture of two stereoisomers of an unsymmetrical diol. The method includes the step of contacting the mixture of two stereoisomers of the unsymmetrical diol with a catalyst of the formula:

in which:
R₁ is H, C₁-C₆ alkyl, aryl; or heteroaryl;
R₂ is, independently for each occurrence, C₁-C₆ alkyl, aryl, or heteroaryl;
Y is O, S or N—R₃;
Z is, independently for each occurrence, O or S;
R₃ is H, C₁-C₆ alkyl, aryl, or heteroaryl;
R₄ is (CH₂)ₘ—X;
X is aryl or heteroaryl;
m is 0, 1 or 2; and
n is an integer from 1 to 10;

or a salt thereof;

in the presence of a silylating reagent and a base; under conditions such that a hydroxy group of one of the stereoisomers of the unsymmetrical diol is selectively silylated compared to another stereoisomer of the unsymmetrical diol.

In certain embodiments, the unsymmetrical diol is a 1,2-diol, or a 1,3-diol. In certain embodiments, the unsymmetrical diol is a triol.

In certain embodiments, the base is an organic base.

In certain embodiments, the catalyst is represented by the formula:

wherein:

R₂ is a straight or branched alkyl group;

R₅ is an aryl or alkyl group;

R₆ is a lower alkyl;

R₇ is a lower alkyl; and

n=1, 2, 3, 4, 5, 6, 7, 8, 9, or 10;

or a salt thereof.

In certain embodiments, the catalyst is represented by the formula:

wherein:

R₅ is phenyl or t-butyl; and R₈ and R₉ are each independently H, alkyl or aryl;

or a salt thereof.

In certain embodiments, R₈ and R₉ are each independently selected from methyl and ethyl. In certain embodiments, R₈ and R₉ are each methyl.

In another aspect, the invention provides a system or a reaction mixture comprising:

(i) a catalyst of the formula:

in which:
R₁ is H, C₁-C₆ alkyl, aryl, or heteroaryl;
R₂ is, independently for each occurrence, C₁-C₆ alkyl, aryl, or heteroaryl;
Y is O, S or N—R₃;
Z is, independently for each occurrence, O or S;
R₃ is H, C₁-C₆ alkyl, aryl, or heteroaryl;
R₄ is (CH₂)ₘ—X;
X is aryl or heteroaryl;
m is 0, 1 or 2; and
n is an integer from 1 to 10;

or a salt thereof;

(ii) a silylating reagent; and

(iii) a base.

In certain embodiments, the system or reaction mixture further includes a solvent. In certain embodiments, the system or reaction mixture further includes a 1,2-diol or a 1,3-diol.

In certain embodiments, the base is an organic base.

### BRIEF DESCRIPTION OF THE DRAWINGS

The invention will be further described with reference to the attached drawings.

FIG. 1 shows a general representation of an silylation reaction of a 1,2-diol.

FIG. 2A shows an overview of initial screens on meso-1,3-cyclopentenediol. FIG. 2B shows conditions for an embodiment of the invention.

FIG. 3 shows an embodiment of a reaction scheme for producing the catalysts of the invention.

FIG. 4 shows the effect of modification of the amino acid portion of the catalyst on catalytic activity.

FIG. 5 shows the effect of certain modifications of the N-terminal portion of the catalyst on catalytic activity.

FIG. 6 shows the effect of modification of the C-terminal portion of the catalyst on catalytic activity.

FIG. 7 shows additional data on the effect of modification of the C-terminal portion of the catalyst on catalytic activity.

FIG. 8 shows various embodiments and analogs of the catalysts of the invention.

FIG. 9 shows an overview of a kinetic resolution test and a working model for the transition state of the reaction.

FIG. 10 shows the results of silylation reactions on various 1,3-diol substrates.

FIG. 11 shows the results of silylation reactions on various 1,2-diol substrates.

FIG. 12 shows representations of a kinetic resolution of diols according to the invention.

While the above-identified drawings set forth illustrative embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the invention.

### DETAILED DESCRIPTION OF THE INVENTION

The invention provides methods and compositions for direct asymmetric silylation of a substrate to provide enantiomerically enriched silylated products. The presently disclosed methods and systems utilize one of a family of catalysts of the invention. A benefit of the invention is the ability to bypass the need to access chiral materials through acyl intermediates. This step saving process will be of interest to those involved in the asymmetric synthesis of complex molecules.

In certain aspects, the invention relates to a reaction wherein a substrate (for example, polyhydroxy compound such as a diol) is reacted with a silylating agent in the presence of a catalyst of the invention to effect direct asymmetric silylation of the substrate. In certain embodiments, the reaction takes place in the presence of an organic base. In certain embodiments, the reaction takes place in the presence of silylating agents. Further, the invention provides systems for reacting particular catalysts, silylating agents, solvents at a desired reaction temperature to produce a desired product.

In certain embodiments, the invention provides additional opportunities for reagent control in the silylation process. Whereas silylation reactions are typically under substrate control, the asymmetric silylation catalysts of the invention may offer new forms of reagent control in the silylation reaction. In addition, the catalyst may override a substrate's inherent reactivity profile with the reactivity preference of the catalyst. This type of control over site selectivity is of fundamental importance in the development of short and efficient synthesis of molecules of value.

The invention further provides a collection of novel, silaphilic systems useful for effecting and controlling other group transfer reactions.

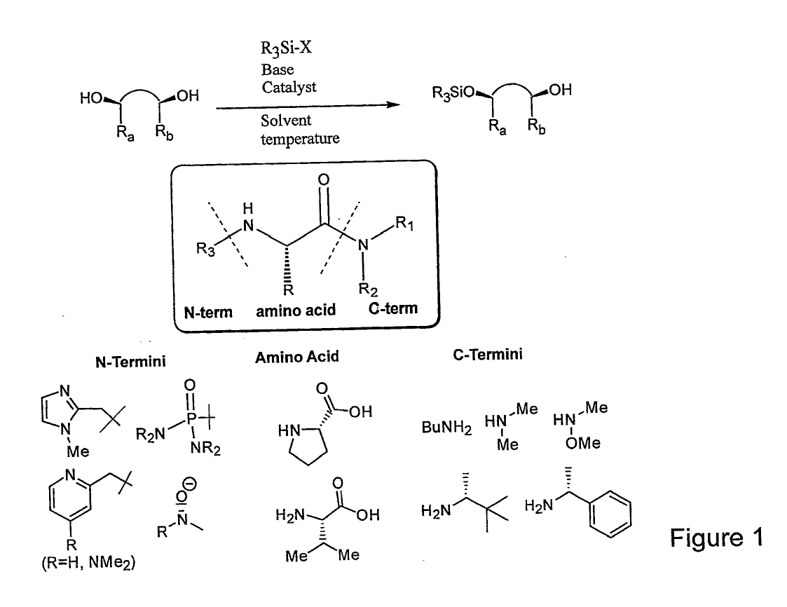

FIG. 1 shows a general reaction scheme of a silylation reaction according to the invention. As shown, certain embodiments relate to a reaction wherein a substrate, in this case a diol, is reacted with a silylating agent in the presence of a catalyst to produce direct asymmetric silylation of the substrate, that is, silylation of the substrate to produce an optically enriched silylated product, without requiring additional synthetic steps or intermediates. In certain embodiments, the reaction takes place in the presence of an organic base. Each of these components (the substrate, the catalyst, silylating agent and organic base) are discussed in more detail below. Further, the invention provides a system for reacting particular substrates, catalysts, silylating agents at a desired reaction temperature (optionally in solvents) to produce a desired product. The experimental section below and the accompanying Figures illustrate examples of these various components and systems of combining the various components to produce a desired product.

### DEFINITIONS

As used herein, the term “alkyl” (either alone or as part of another term, e.g., alkylaryl, alkylamino, alkylthio, alkoxy and the like) means a saturated straight chain or branched hydrocarbon typically having from 1 to 10 carbon atoms. Representative saturated straight chain alkyls include -methyl, -ethyl, -n-propyl, -n-butyl, -n-pentyl, -n-hexyl, -n-heptyl, -n-octyl, -n-nonyl and -n-decyl. Saturated branched alkyls include -isopropyl, -sec-butyl, -isobutyl, -tert-butyl, -isopentyl, -2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. Alkyl groups included in compounds of this invention may be optionally substituted with one or more conventionally used substituents, such as amino, alkylamino, alkoxy, alkylthio, oxo, halo, acyl, nitro, hydroxyl, cyano, aryl, alkylaryl, aryloxy, arylthio, arylamino, cycloalkyl, cycloalkyloxy, cycloalkylthio, cycloalkylamino, heterocyclyl, heterocyclyloxy, heterocyclylamino, heterocyclylthio, and the like. In addition, a carbon in the alkyl segment, typically an internal carbon atom in an alkyl segment, may be substituted with carbonyl (C═O), thiocarbonyl (C═S), oxygen (O), sulfur (S), or nitrogen (N). Lower alkyls (an alkyl group having 1 to 6 carbon atoms in the alkyl backbone) are typically preferred for the compounds of this invention.

As used herein, the term “cycloalkyl” (either alone or as part of another term) means a saturated cyclic alkyl radical typically having from 3 to 10 carbon atoms. Representative cycloalkyls include cyclopropyl, cyclobutyl, cyclopentyl, cyclohexyl, cycloheptyl, cyclooctyl, cyclononyl, and cyclodecyl. A cycloalkyl group may optionally be substituted with substituents as described for an alkyl group.

As used herein, a “diol” is an organic compound having at least two hydroxyl (—OH) groups attached to two different saturated carbon atoms. Similarly, a “triol” is an organic compound having at least three hydroxyl (—OH) groups attached to three different saturated carbon atoms. A polyhydroxyl compound has multiple (e.g., 2, 3, 4, 5, 6, etc.) hydroxyl groups attached to multiple different saturated carbon atoms.

As used herein, the term “heterocyclyl” (either alone or as part of another term) means a cyclic group as for a cycloalkyl, in which one or more carbon atoms of the cycloalkyl moiety is/are replaced by a heteroatom, e.g., O, S, or N. A heterocyclyl group typically has from 3 to 10 atoms in the heterocyclic ring. Exemplary heterocyclyl groups include pyrrolidinyl, piperidinyl, piperazinyl, tetrahydrofuranyl, tetrahydropyranyl, and tetrathydrothiophene.

As used herein, the term “aryl” (either alone or as part of another term, e.g., alkylaryl, aryloxy, arylamino and the like) means a monocyclic or polycyclic-aromatic ring or ring radical comprising carbon and hydrogen atoms. Examples of aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more conventional aryl substituents (including without limitation alkyl (including lower alkyl), hydroxy, alkoxy (including lower alkoxy), alkylthio, cyano, halo, amino, and nitro). In certain embodiments, the aryl group is a 6-membered carbocyclic ring.

As used herein, the term “heteroaromatic” or “heteroaryl” (either alone or as part of another term) means a monocyclic or polycyclic heteroaromatic ring (or radical thereof) comprising carbon atom ring members and one or more heteroatom ring members (such as, for example, oxygen, sulfur or nitrogen). In one embodiment, the heteroaromatic ring is selected from 5-8 membered heteroaryl rings. In another embodiment, the heteroaromatic ring is a 5 or 6 membered ring. Representative heteroaryls include furyl, thienyl, pyrrolyl, oxazolyl, imidazolyl, indolizinyl, thiazolyl, isoxazolyl, pyrazolyl, isothiazolyl, pyridinyl, pyridazinlyl, pyrazinlyl, triazolyl, thiadiazolyl, benzofuryl, benzothienyl, indolyl, isoindolyl, benzoxazolyl, benzimidazolyl, benzothiazolyl, isoxazolyl, indazolyl, benzoisothiazolyl, benzopyrazinyl, benzotriazolyl, benzothiadiazolyl, quinolyl, isoquinolyl, quinazolyl, phthalazolyl, cinnolyl, and the like. Heteroaryl groups may be optionally substituted with one or more substituents including (but not limited to) alkyl, cycloalkyl, amino, alkylamino, alkoxy, alkylthio, oxo, halo, acyl, nitro, hydroxyl, cyano, aryl, alkylaryl, aryloxy, arylthio, arylamino, cycloalkyloxy, cycloalkylthio, cycloalkylamino, heterocyclyl, heterocyclyloxy, heterocyclylamino, heterocyclylthio, and the like.

As used herein, the term “halogen”, “halo” (either alone or as part of another term) means —F, —Cl, —Br or —I.

The compounds of the invention may contain one or more chiral centers and/or double bonds and, therefore, exist as stereoisomers, such as double-bond isomers (i.e., geometric isomers), enantiomers, or diastereomers. With respect to each compound or generic formula depicted herein by structure or by name, unless the stereochemistry at a particular atom is defined, it is understood that all of the corresponding enantiomers and stereoisomers, that is, both the stereomerically pure form (e.g., geometrically pure, enantiomerically pure, or diastereomerically pure) and enantiomeric and stereoisomeric mixtures, are included.

### Catalyst:

In one aspect, the present invention provides novel catalysts useful for effecting a variety of organic transformations. For example, the invention provides a novel family of catalysts which allow for direct asymmetric silylation of a substrate to provide enantiomerically enriched silylated products.

In certain embodiments, the catalysts of the invention are structurally related to natural or unnatural amino acids, and, in certain embodiments, can be derived or prepared from α-amino acids. In certain embodiments, the catalysts are structurally modular, thereby providing a robust method of preparing various catalyst candidates by variations in the identity of amino acid constituent and also by incorporation of additional structural features through the amide bond linkage.

In general, the catalyst will contain at least one Lewis basic moiety that, through association, can increase the electrophilicity of a silylation reagent (Lewis base activation). Lewis basic silaphilic moieties, e.g., phosphoramide, N-oxides or heterocycles (such as N-methylimidazole) can be incorporated into the catalyst as substituents on the N-terminus of the amino acid. In addition, the amide portion in the catalyst is another silaphilic site. The combination of these moieties could help, at least in part, confer upon the catalyst the ability to activate silicon species and facilitate the reaction. A wide range of catalysts may be produced. Various embodiments of catalysts are disclosed below; those skilled in the art will recognize that various other catalysts not specifically disclosed below are clearly within the spirit and scope of the present invention.

In certain embodiments, a compound or catalyst of the invention may be represented by the following formula (Formula I):

in which:
R₁ is H, C₁-C₆ alkyl, aryl, or heteroaryl;
R₂ is, independently for each occurrence, C₁-C₆ alkyl, aryl, or heteroaryl;
Y is O, S or N—R₃;
Z is, independently for each occurrence, O or S;
R₃ is H, C₁-C₆ alkyl, aryl, or heteroaryl;
R₄ is (CH₂)ₘ—X;
X is aryl or heteroaryl;
m is 0, 1 or 2; and
n is an integer from 1 to 10;

or a salt thereof.

In certain embodiments, n is 1. In certain embodiments, Y is N—R₃. In certain embodiments, R₃ is H. In certain embodiments, Z is O for each occurrence. In certain embodiments, m is 1. In certain embodiments, X is heteroaryl; in certain embodiments, X is a 5-membered nitrogen-containing heteroaryl; in certain embodiments, X is 1-methylimidazol-2-yl. In certain embodiments, R₁ is —CHQ₁Q₂, in which Q₁ and Q₂ are independently alkyl, cycloalkyl, or aryl. In certain embodiments, Q₁ is alkyl and Q₂ is phenyl, 1-naphthyl, 2-naphthyl, or tetrahydronaphth-1-yl; in other embodiments, Q₂ is methyl, isopropyl or t-butyl. In certain embodiments, Q₁ is methyl, isopropyl, or t-butyl.

In certain embodiments, R₂ is selected from methyl (e.g., corresponding to the side-chain of alanine), isopropyl (valine), t-butyl, 2-methyl-propan-1-yl (isobutyl, leucine), 2-butyl (isoleucine or allo-isoleucine), 1-benzyloxy-ethan-1-yl, 1-butyloxy-ethan-1-yl, cyclohexyl, CH₂-cyclohexyl, or phenyl.

When Y is O, then in certain embodiments, R₁ is C₁-C₆ alkyl, e.g., t-butyl.

In certain embodiments, a compound or catalyst of the invention can be represented by the structure of Formula II:

in which

R₁ is H, C₁-C₆ alkyl, aryl, or heteroaryl;

R₂ is C₁-C₆ alkyl, aryl, or heteroaryl;

R₃ is H, C₁-C₆ alkyl, aryl, or heteroaryl;

R₄ is (CH₂)ₘ—X;

X is aryl or heteroaryl;

m is 0, 1 or 2; and

n is an integer from 1 to 10;

or a salt thereof

In certain embodiments, the compound of Formula II can be represented by the following structure (Formula IIa)

in which R₁, R₂, R₃, R₄, and n are as described for Formula II.

Although the structure of Formula IIa provides for a specific configuration at the carbon to which R₂ is attached, it will be appreciated that, unless a specific stereoisomer is indicated, the invention provides both the indicated stereoisomer, the opposite stereoisomer, and mixtures (e.g., a racemic mixture) of the two stereoisomers.

Thus, for example, in certain embodiments of the compound of Formula II or Formula IIa:

R₁ is H, C₁-C₆ alkyl, aryl, or heteroaryl;

R₂ is C₁-C₆ alkyl, aryl, or heteroaryl;

R₃ is H, C₁-C₆ alkyl, aryl, or heteroaryl;

R₄ is (CH₂)ₘ—X;

X is aryl or heteroaryl;

m is 0, 1 or 2; and

n is an integer from 1 to 10;

or a salt thereof.

In certain embodiments, R₁ is H, C₁-C₆ alkyl or aryl. In certain embodiments, R₁ is H, methyl or tert-butyl.

In certain embodiments, R₂ is a side chain of a naturally-occurring amino acid (e.g., methyl, isopropyl). In certain embodiments, R₂ is a side chain of a non-naturally-occurring amino acid (e.g., t-butyl, cyclohexyl, CH₂-cyclohexyl, phenyl).

In certain embodiments, R₃ is H, C₁-C₆ alkyl or aryl. In certain embodiments, R₃ is H, methyl or tert-butyl.

In certain embodiments, one of R₁ and R₃ is H.

In certain embodiments, m is 1.

In certain embodiments, n is 1.

In certain embodiments, X is heteroaryl, such as a nitrogen-containing 5- or 6-membered heteroaryl group, including, e.g., a substituted or unsubstituted imidazolyl group, pyridinyl group, oxazolyl group, thiazolidinyl group, pyrazinyl group. In certain embodiments, X is an imidazol-2-yl group, preferably substituted with a lower alkyl group on a nitrogen of the imidazole ring (e.g., an N-methylimidazol-2-yl group).

In the compounds of Formula I and Formula II, R₁, R₂, R₃, R₄ and n represent a wide range of possible substituents. The various figures and tables in this disclosure provide various embodiments of the presently disclosed catalysts comprising various substituents. As an example, in certain embodiments, n=1, 2, 3, 4, 5, 6, 7, 8, 9, or 10. In certain embodiments, n may be greater than 10.

In certain embodiments, the catalyst can be represented by Formula III

in which:

R₂ is a straight or branched alkyl group;

R₅ is an aryl or alkyl group (such as phenyl or t-butyl);

R₆ is a lower alkyl;

R₇ is a lower alkyl; and

n=1, 2, 3, 4, 5, 6, 7, 8, 9, or 10;

or a salt thereof.

In certain embodiments, the compound of Formula III can be represented by the following structure (Formula IIIa):

in which R₂, R₅, R₆, R₇ and n are as described for Formula III.

Although the structure of Formula IIIa provides for a specific configuration at the carbon to which R₂ and R₆ are attached, it will be appreciated that, unless a specific stereoisomer is indicated, the invention provides both the indicated stereoisomer, the opposite stereoisomer, and mixtures (e.g., a racemic mixture) of the two stereoisomers. Thus, the chirality of the catalyst can be selected to provide a desired chirality of the silylated reaction product.

In certain embodiments of Formula III or IIIa:

R₂ is a straight or branched alkyl group;

R₅ is phenyl or t-butyl;

R₆ is a lower alkyl;

R₇ is a lower alkyl (preferably methyl); and

n=1, 2, 3, 4, 5, 6, 7, 8, 9, or 10;

or a salt thereof.

In still further embodiments, the catalyst may be represented by the formula (Formula IV):

in which

R₅ is alkyl (such as t-butyl) or aryl (such as phenyl); and

R₈ and R₉ are each independently H, alkyl or aryl;

or a salt thereof.

In certain embodiments, the compound of Formula IV can be represented by the following structure (Formula IVa):

wherein R₅, R₈ and R₉ are as described for Formula IVa.
R₈ and R₉ represent alkyl groups (preferably lower alkyl groups); and R₅ represents:

Although the structure of Formula IVa provides for a specific configuration at the carbon to which the atoms bearing R₈ and R₉, and R₅, are attached, it will be appreciated that, unless a specific stereoisomer is indicated, the invention provides both the indicated stereoisomer, the opposite stereoisomer, and mixtures (e.g., a racemic mixture) of the two stereoisomers.

In certain embodiments of Formulae IV and IVa, R₈ and R₉ represent alkyl groups (preferably lower alkyl groups); and R₅ represents phenyl or t-butyl.

In certain embodiments, the catalyst has the formula:

in which A, B, and C are as shown in the Table below:

| Entry | A | B | C |
|---|---|---|---|
| 1 | (1-methyl-1H- | 2-propyl | 1-phenylethyl |
| | imidazol-2-yl)-CH₂— | | |
| 2 | (1-methyl-1H- | 2-propyl | 3,3-dimethylbutan-2-yl |
| | imidazol-2-yl)-CH₂— | | |
| 3 | methyl | 2-butyl | 3,3-dimethylbutan-2-yl |
| 4 | ethyl | 2-butyl | 3,3-dimethylbutan-2-yl |
| 5 | phenyl | 2-butyl | 3,3-dimethylbutan-2-yl |
| 6 | (1-methyl-1H- | 2-propyl | n-butyl |
| | imidazol-2-yl)-CH₂— | | |
| 7 | (1-methyl-1H- | 2-propyl | benzyl |
| | imidazol-2-yl)-CH₂— | | |
| 8 | (1-methyl-1H- | t-butyl | 3,3-dimethylbutan-2-yl |
| | imidazol-2-yl)-CH₂— | | |
| 9 | (1-methyl-1H- | isobutyl | 3,3-dimethylbutan-2-yl |
| | imidazol-2-yl)-CH₂— | | |
| 10 | (1-methyl-1H- | 2-butyl | 2-butyl |
| | imidazol-2-yl)-CH₂— | | |
| 11 | (1-methyl-1H- | cyclohexyl | 3,3-dimethylbutan-2-yl |
| | imidazol-2-yl)-CH₂— | | |
| 12 | (1-methyl-1H- | (cyclohexyl)- | 3,3-dimethylbutan-2-yl |
| | imidazol-2-yl)-CH₂— | CH₂— | |
| 13 | (1-methyl-1H- | phenyl | 3,3-dimethylbutan-2-yl |
| | imidazol-2-yl)-CH₂— | | |
| 14 | (1-methyl-1H- | 1-butyloxy- | 3,3-dimethylbutan-2-yl |
| | imidazol-2-yl)-CH₂— | ethan-1-yl | |
| 15 | (1-methyl-1H- | 1-benzyloxy- | 3,3-dimethylbutan-2-yl |
| | imidazol-2-yl)-CH₂— | ethan-1-yl | |
| 16 | (1-phenyl-1H- | 2-butyl | 3,3-dimethylbutan-2-yl |
| | imidazol-2-yl)-CH₂— | | |
| 17 | (1-methyl-1H- | 2-butyl | 3,3-dimethylbutan-2-yl |
| | imidazol-2-yl)- | | |
| | CH(CH₃)— | | |
| 18 | (1-methyl-1H- | 2-butyl | 3,3-dimethylbutan-2-yl |
| | imidazol-2-yl)-CH₂— | | |

In certain embodiments, the catalyst can be represented by the formula:

in which R₁, R₄, Y and Z are as described for Formula I.

In certain embodiments, a catalyst of the invention is provided as the free base form. In other embodiments, the catalyst is provided as a salt form. Salts of the catalysts of the invention include acid addition salts (e.g., hydrochloride, phosphate, sulfate, and the like), and, for those catalysts having an acid functionality (such as a carboxylate) as part of the catalyst structure, base addition salts (e.g., sodium, potassium, ammonium, and the like).

As stated above, these embodiments are merely illustrative of the family of catalysts within the spirit and scope of the present invention. The Experimental Section to follow lists various other embodiments of catalysts which are within the spirit and scope of the present invention. In addition, the accompanying Figures disclose a wide range of embodiments of catalysts which are within the spirit and scope of the present invention. Those skilled in the art will recognize that various additional catalysts are within the spirit and scope of the present invention.

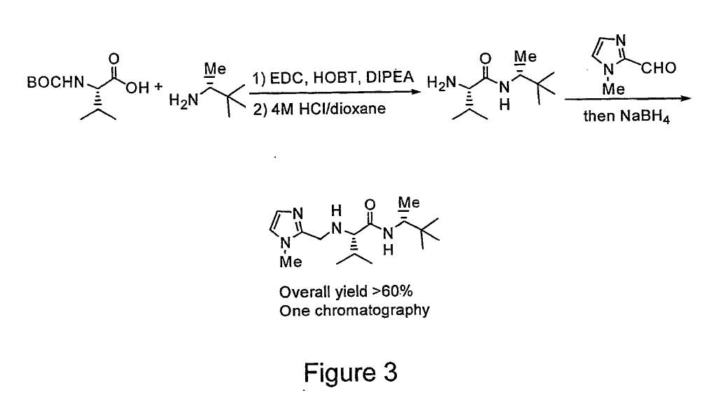

The catalysts of the invention can be prepared according to a variety of methods, some of which are known in the art. For example, as illustrated in FIG. 3, an N-protected amino acid (e.g., an N-Boc amino acid) can be coupled with an amine using standard amide-forming conditions (e.g., coupling agents such as ethyl-3-(3-dimethylaminopropyl)-carbodiimide (EDC)), followed by N-deprotection (e.g., under acidic conditions) to provide an amino acid amide. The free amino group of this amino acid amide can be further derivatized, e.g., by reductive amination with an aldehyde in the presence of a reducing agent such as sodium borohydride, or alternatively by N-alkylation (e.g., by reaction with an alkyl halide), to provide a catalyst according to the invention. See, e.g., Example 1, infra. Other methods for preparing the catalysts of the invention will be apparent to the skilled artisan in view of the present disclosure.

In certain embodiments, the catalyst can be used at about a 20% molar ratio to the substrate, although the amount of catalyst can be adjusted to provide faster reaction times (generally using more catalyst) or more selective conversion of the substrate to the desired product (generally using less catalyst). Thus, in certain embodiments, the amount of catalyst used can be, e.g., 0.05%, 0.1%, 0.5%, 1%, 2%, 5%, 10%, 15%, 20%, 25% or 30% (mol:mol) based on the amount of substrate.

In certain embodiments, a catalyst according to the invention is inexpensive and simple to produce, and preferably is stable in air. In addition, in certain preferred embodiments, the catalyst can generally be recovered after completion of a reaction (e.g., by an acidic workup), and can be reused without noticeable decrease in efficiency.

### Substrates:

The invention provides methods and compositions useful in silylation reactions, e.g., the direct asymmetric silylation of a substrate to provide enantiomerically enriched silylated products. In certain embodiments, the substrate is an alcohol. In certain embodiments, the alcohol is a polyhydroxyl compound (i.e., the compound has multiple hydroxyl groups). In certain embodiments, the substrate is a diol. In certain embodiments, the substrate is a 1,2 diol, including a cyclic 1,2-diol. In certain embodiments, the substrate is a cyclic 1,3 diol. In certain embodiments, the substrate is an acyclic 1,3 diol. In certain embodiments, the substrate is a 1,4 diol. In certain embodiments, the substrate is a primary diol. In certain embodiments, the substrate is an unsymmetrical diol (e.g., a 1, 2 or 1,3-diol). In certain embodiments, an unsymmetrical diol can be represented by the formula:

in which Rₐ and R_{b} are different and are independently selected from, e.g., alkyl, cycloalkyl, heterocyclyl, aryl, heteroaryl, and the like; or Rₐ and R_{b} can be joined to form a cyclic moiety (e.g., a cyclic 1,2-diol or 1,3-diol).

In certain embodiments, the substrate is an amine. In an embodiment, the substrate is a carboxylic acid. In certain embodiments, the substrate is a secondary alcohol. In certain embodiments, the substrate is a tertiary alcohol. In certain embodiments, the substrate comprises a ketone group. In certain embodiments, the substrate is a hydroxy ketone, such as a beta-hydroxy ketone, e.g., a compound represented by the formula:

in which Rₐ and R_{b} are the same or different and are independently selected from, e.g., alkyl, cycloalkyl, heterocyclyl, aryl, heteroaryl, and the like; or Rₐ and R_{b} can be joined to form a cyclic moiety (e.g., a cyclic beta-hydroxyketone).

Those skilled in the art will recognize that various other substrates are within the spirit and scope of the present invention.

### Silylating Agents:

As described herein, the presently invention provides methods, compositions, and systems for silylation (e.g., direct asymmetric silylation) of a substrate in the presence of a silylating agent and a catalyst to provide silylated products (which in certain embodiments may be enantiomerically enriched). A silylating agent can be any agent capable of reacting with an alcohol or other functional group to provide a silyl ether (e.g., capable of reacting with a hydroxyl group of an organic compound having at least one hydroxyl group), and can generally be represented by the formula R′R″R′″SiZ. In general, Z can be a leaving group suitable for providing a reactive silylating reagent for an alcohol, e.g., Z can be Cl, L, Br, OTf, imidazole, phenol, acetamide, formamide, cyano, and the like. The groups R′, R″, and R′″ are individually selected from alkyl, alkenyl, or aryl groups. For example, in one embodiment, the silylating agent is trimethyl silyl-Z (TMS-Z). In another embodiment, the silylating agent is triethyl silyl-Z (TES-Z). In additional embodiments, the silylating agent is: t-butyl dimethylsilyl-Z (TBS-Z); t-butyldiphenylsilyl-Z (TBDPS-Z); triisopropylsilyl-Z (TIPS-Z); allyl-dimethylsilane-Z; or allyl-tert-butylmethyl silane-Z. Those skilled in the art will recognize that a silylating agent can include various other silyl moieties and various other substituents represented by Z without departing from the spirit and scope of the present invention.

In certain embodiments, in a desymmetrization reaction, a silylating agent will be used at about a 1:1 molar ratio to the substrate, although an excess of silylating agent (e.g., 1.1, 1.2, 1.25, 1.3, 1.4, 1.5 or 2 equivalents) may be used to ensure complete reaction at one (but generally only one) hydroxyl group of the substrate. However, for a kinetic resolution, the ratio of silylating agent to substrate may be less than 1:1 (e.g., 0.5 equivalents, 0.6 equivalents, 0.75 equivalents, etc., of silylating agent to 1 equivalent of substrate) in order to ensure kinetic resolution and conversion to substantially the desired product.

### Base:

In certain embodiments of the methods of the invention, a base is used together with a silylating reagent and a catalyst of the invention to provide enantiomerically enriched silylated products. In one embodiment, the base is an organic base, such as a basic amine. In one embodiment, the organic base is diisopropylethylamine (DIPEA). In certain embodiments, the organic base is selected from a group consisting of Et₃N, pyridine, and lutidine. In other embodiments, the base is an inorganic base, e.g., K₂CO₃, or Na₂CO₃. Those skilled in the art will recognize that various bases, e.g., organic bases, may be used in the methods of the invention.

In certain embodiments, in a desymmetrization reaction, a base will be used at about a 1:1 molar ratio to the substrate, although an excess of base (e.g., 1.1, 1.2, 1.25, 1.3, 1.4 or 1.5 equivalents) may be used to ensure complete reaction at one (but generally only one) hydroxyl group of the substrate. However, for a kinetic resolution, the ratio of base to substrate may be less than 1:1 (e.g., 0.5 equivalents, 0.6 equivalents, 0.75 equivalents, etc., of base to 1 equivalent of substrate) in order to ensure kinetic resolution and conversion to substantially the desired product.

### Solvent:

In certain embodiments, the silylation reaction is performed in a solution. A solvent can be selected according to considerations known in the art, e.g., a solvent should solubilize at least some of the reaction components without causing undesirable side reactions. In certain embodiments, the solvent is a non-protic solvent such as tetrahydrofuran (THF) or toluene.

In another aspect, the invention provides a system or a reaction mixture comprising:

(i) a catalyst of the invention according to any of the Formulae herein;

(ii) a silylating reagent; and

(iii) a base.

In certain embodiments, the system or reaction mixture further includes a solvent. In certain embodiments, the system or reaction mixture further includes a substrate (e.g., a 1,2-diol, 1,3-diol, or a beta-hydroxy ketone.

In certain embodiments, the base is an organic base.

### Methods of the Invention

In certain aspects, the invention provides novel catalysts and methods for silylation of a substrate, e.g., desymmetrization of a substrate via direct asymmetric silylation. In some cases, the method is highly enantioselective for a wide range of substrates.

In one aspect, the invention provides a method for direct asymmetric silylation of an alcohol. The method includes the step of reacting the alcohol with a silylating agent in the presence of a catalyst, wherein an enantiomerically enriched silylated product is produced in a single step. The catalyst can be a chiral catalyst and can be a compound according to any of the formulae disclosed herein. In certain embodiments, the alcohol is a diol, such as a 1,2-diol (including a 1,2 cyclic diol) or a 1,3-diol (including a 1,3 cyclic diol).

In another aspect, the invention provides a method of silylating a hydroxy group of a compound comprising at least one hydroxy group. The method includes the step of contacting the compound with a catalyst of the invention, in the presence of a silylating reagent and a base, under conditions such that a hydroxy group of the compound is silylated. In certain embodiments, the compound is a diol, such as a 1,2-diol or a 1,3-diol. In certain embodiments, the compound is a triol. In certain embodiments, the compound is a beta-hydroxy ketone. In certain embodiments, the base is an organic base.

In another aspect, the invention provides a method of kinetic resolution of a mixture of two stereoisomers of an unsymmetrical diol. The method includes the step of contacting the mixture of two stereoisomers of the unsymmetrical diol with a catalyst of the invention, in the presence of a silylating reagent and a base; under conditions such that a hydroxy group of one of the stereoisomers of the unsymmetrical diol is selectively silylated compared to another stereoisomer of the unsymmetrical diol. In certain embodiments, the unsymmetrical diol is a 1,2-diol, or a 1,3-diol. In certain embodiments, the unsymmetrical diol is a triol.

In certain embodiments, the base is an organic base.

There are additional methods that become possible with the asymmetric silylation reaction. One exciting aspect is the under-explored opportunity to introduce and use the asymmetric silylation in subsequent transformations. As an example, the asymmetric allylsilylation may be combined with known processes, such as olefin metathesis, to provide rapidly novel chiral building blocks.

The opportunity to introduce a chiral silyl group directly should also become possible with these asymmetric catalysts. When protecting a hydroxyl group, reagents are typically used such that a new silicon stereogenic center is not generated during the silylation reaction. With these new catalysts, it may be possible to control the absolute silicon stereochemistry in the silylation event. A temporary, asymmetric silyl group can then, in turn, be used as a chiral auxiliary to effect additional asymmetric transformations.

In addition, a catalyst may be identified that can “resolve” racemic trialkylsilylhalides in a hydroxyl silylation event (as described herein). Depending on the relative rates of the various processes and the role of additives, this asymmetric transformation may proceed as a dynamic kinetic resolution using all the substrate in generating the desired product with high enantioselectivity. In certain embodiments, use of an allyldialkylsilane and treatment of the chiral silylated product with a Lewis acid can then lead to further reactions, such as an intramolecular allylation of an aldehyde or ketone functionality (see, e.g., Scheme 8, infra). The nature of the silyl group, however, will have a strong stereochemical influence on the facial selectivity of this process, providing the chiral diol product upon work-up.

The opportunity to carry out reagent-controlled, site selective silylations will also become possible for the first time with an effective asymmetric silylation catalyst. The strategy is illustrated below, where the two catalyst enantiomers are shown to provide complementary site selectivity in the silylation of the diol precursor. Thus, selection of an appropriate catalyst stereoisomer can result in formation of either of two products, allowing for selection of the desired product.

The following experimental section illustrates various embodiments of the invention; including various catalysts for the desymmetrization of substrates via direct asymmetric silylation. The scope of the present disclosure is in no way meant to be limited by the following experimental section; the section is merely illustrative.

### EXPERIMENTAL

### Example 1

### Representative Procedure for Preparation of the Synthesis of Catalyst

### (S)—N—((R)-3,3-dimethylbutan-2-yl)-3,3-dimethyl-2-((1-methyl-1H-imidazol-2-yl)methylamino)butanamide (Compound 9)

Boc-tert-Leucine (2.3 g, 10 mmol) and (R)-3,3-dimethyl-2-butylamine (1.3 mL, 10 mmol) were dissolved in 40 mL CH₂Cl₂ in a 100 mL round bottom flask. To this solution were added EDC (2.1 g, 11 mmol), HOBt (1.7 g, 11 mmol) and DIPEA (4.4 mL, 25 mmol). The reaction was allowed to stir for 16 h at room temperature after which time 15 mL of 10% citric acid was added. The organic layer was separated and washed with 15 mL saturated NaHCO₃ and then 15 mL brine, dried over anhydrous Na₂SO₄, filtered, and concentrated under reduced pressure to yield a white solid. This white solid was placed in a round-bottom flask and cooled to 0° C., HCl/dioxane (7.5 mL of 4.0 M solution) was then added via syringe. The reaction was allowed to warm to room temperature over 1 h and was then concentrated. The crude product was dissolved in water and basified with 3 N NaOH until pH 12. The mixture was extracted with CH₂Cl₂ (3×15 mL), washed with brine (1×10 mL), and then dried over anhydrous Na₂SO₄. After filtration and removal of the solvent the crude amine was dissolved in 5 mL of CH₂Cl₂, followed by the addition of 1-methyl-2-imidazolecarboxaldehyde (1.1 g, 10 mmol) and MgSO₄. The mixture was allowed to stir for 12 h at room temperature, filtered and concentrated to give a white solid. The crude material was dissolved in MeOH and cooled to 0° C. To this solution was added NaBH (1.1 g, 30 mmol) and 2 drops of conc. HCl. The reaction was allowed to stir for 0.5 h at 0° C. and then 1 h at room temperature, after which time saturated NaHCO₃ was added to quench the reaction. The mixture was extracted with CH₂Cl₂ (3×15 mL), washed with brine (1×10 mL), dried over anhydrous Na₂SO₄ and concentrated to yield the crude catalyst as a beige solid. Purification by chromatography (CH₂Cl₂ to 98:2 CH₂Cl₂:MeOH) yielded the catalyst as a white solid (1.9 g, 61%). MP: 130.8-132.0° C. IR: 3362 (br), 3267 (br), 3060 (m), 3025, (m), 2921 (s), 1660 (s), 1366 (w), 1034 (w) cm⁻¹. ¹H NMR (CDCl₃, 400 MHz): δ 6.94 (1H, d, J=1.2 Hz), 6.82 (1H, d, J=1.2 Hz), 6.51 (1H, d, J=10.0 Hz), 3.91 (1H, dq, J=9.6, 6.8 Hz), 3.80 (1H, d, J=14.0 Hz), 3.62 (3H, s), 3.61 (1H, d, J=14.0 Hz), 2.68 (1H, s), 2.15 (1H, br, s), 1.06 (3H, d, J=6.8 Hz), 0.97 (9H, s), 0.92 (9H, s). ¹³C NMR (CDCl₃, 100 MHz): δ 171.9, 146.1, 127.5, 121.2, 72.5, 52.9, 44.8, 34.3, 34.2, 32.9, 27.5, 26.7, 16.8. HRMS (m/z+H): Calculated: 309.2654; Found: 309.2652. Optical Rotation: [α]²⁷_{D} −95 (c=3.0, CHCl₃).

### Example 2

### Desymmetrization of Meso-Diols

To access increasingly complex chiral, non-racemic chemical and pharmaceutical targets in an economically feasible manner requires the continued development of improved asymmetric synthetic methods. A particularly attractive strategy for generating enantiomerically enriched materials is the selective functionalization of symmetrical precursors. In this process, chiral, non-racemic compounds possessing multiple stereocenters can be produced in a single step in high yield through chemical or enzymatic desymmetrizations of readily available meso-materials.

Meso-diols, for example, have been utilized as popular and useful substrates in these type of desymmetrizations. In most cases, the selective functionalization of these systems involve an asymmetric acylation event. While effective for generating the desired chiral products with high enantiopurity, the reactivity of the resulting mono-acylated products limit their utility in complex molecule synthesis. Typically, the acyl moiety installed in the key desymmetrization event needs to be converted to a more robust functionality before being subjected to further manipulations towards the desired target.

An example of this sequence is illustrated above in Scheme 1. The optically active cyclopenten-1,3-diol monoacetate 3, a valuable building block for the synthesis of prostaglandins and carbocyclic nucleosides, is typically prepared through the enzymatic desymmetrization of the corresponding meso-diol 1 or meso-diacetate 2. The product of this desymmetrization is then subjected to two additional steps to provide the desired silylated non-racemic chiral product 4. Clearly, it would be much more desirable in this case to generate 4 directly through an asymmetric silylation; the present invention provides such a method.

### General Procedure for the Desymmetrization of Meso-Diols by Asymmetric Silylation

Catalyst ((S)—N—((R)-3,3-dimethylbutan-2-yl)-3,3-dimethyl-2-((1-methyl-1H-imidazol-2-yl)methylamino)butanamide, see Example 1) and meso-diol were weighed into a 10×75 mm test tube. DIPEA was then added with a Gilson Pipetman. The contents were dissolved in THF (or PhMe), capped with a septa, and cooled to the appropriate temperature (see below for details) using a cryocool apparatus. TBSCl was dissolved in THF (or PhMe), cooled to the same temperature and then added to the test tube with a Gilson Pipetman. The test tube was capped with a septa, wrapped with Teflon tape and the reaction was allowed to stir for the reported period of time. The reaction was quenched with DIPEA (1.0 equiv. relative to substrate) followed by methanol (10 drops). The mixture was allowed to warm to room temperature and diluted with CH₂Cl₂ (15 mL) and washed with 10% citric acid (20 mL). The aqueous layer was extracted with CH₂Cl₂ (2×15 mL) and the combined organic layer was dried over MgSO₄, filtered and concentrated to afford the crude product as a yellow oil. The product was purified by silica gel chromatography and analyzed for enantioenrichment by chiral GLC (Supelco Beta, or Gamma Dex 120). The aqueous layer was basified with 3 N NaOH until pH 12 and extracted with CH₂Cl₂ (3×15 mL). The combined organic layer was dried over MgSO₄, filtered and concentrated to provide the recovered catalyst as a white solid (mass recovery >95%).

(2R,4S)-4-(tert-Butyl-dimethyl-silanyloxy)-cyclopent-2-enol. The general procedure was followed. Catalyst ((S)—N—((R)-3,3-dimethylbutan-2-yl)-3,3-dimethyl-2-((1-methyl-1H-imidazol-2-yl)methylamino)butanamide, see Example 1) (30.8 mg, 0.100 mmol) and cis-cyclopentene-1,3-diol (50.0 mg, 0.500 mmol) were weighed into a 10×75 mm test tube. DIPEA (109 uL, 0.625 mmol) was then added into the test tube with a 200 uL Gilson Pipetman. The contents were dissolved in 400 uL THF, capped with a septa, and cooled to −78° C. TBSCl (151 mg, 1.00 mmol) was dissolved in 350 uL THF to make the total volume around 500 uL, cooled to −78° C., and then added to the test tube with a 1000 uL Gilson Pipetman. The test tube was capped with a septa, wrapped with Teflon tape and the reaction was allowed to stir at −78° C. for 120 h. After workup as in general procedure, the product was purified by silica gel chromatography (Hexane to 2:1 Hexane:CH₂Cl₂) to yield a pale yellow oil (58 mg, 54% yield). IR (neat, thin film): 3358 (br), 3062 (w), 2961 (m), 2930 (m), 2886 (w), 2860 (m), 1476 (w), 1375 (s), 1262 (s), 1080 (s), 1023 (w), 909 (s), 840 (s), 784 (s), 677 (m) cm⁻¹. ¹H NMR (CDCl₃, 400 MHz): ~ 1H, d, J=5.6 Hz), 5.88 (1H, d, J=5.6 Hz), 4.65 (1H, m), 4.58 (1H, m), 2.68 (1H, dt, J=14.0, 7.2 Hz), 1.82 (1H, d, J=8.8 Hz), 1.51 (1H, dt, J=13.6, 4.8 Hz), 0.89 (9H, s), 0.08 (6H, s). ¹³C NMR (CDCl₃, 100 Mz): δ 137.1, 135.6, 75.4, 75.3, 45.0, 26.2, 18.5, −4.24. Anal Calcd for C₁₁H₂₂O₂Si: C, 61.63; H, 10.34. Found C, 61.57; H, 10.19. Optical Rotation: [α]²⁵_{D} −21 (c=1.0, CHCl₃).

### Example 3

### Catalyst Screening

In this experimental section, a successful desymmetrization of meso-diols through a catalytic asymmetric silylation is disclosed. These studies were initiated by screening representative peptidyl derivatives possessing nucleophilic (silaphilic) functionality for catalytic activity in the asymmetric silylation of cyclopenten-1,3-diol 1 (Scheme 2). For practical purposes, these survey reactions were run in THF at room temperature with tert-butyl-dimethyl-silylchloride (TBSCl) as the silyl source and diisopropylethylamine (DIPEA) as the base. While most of the catalysts showed no reactivity or selectivity, the valine-derived catalysts 5 and 6, which bear N-methyl-imidazole at the N-termini and (R)-phenyl-ethyl-amine or (R)-3,3-dimethyl-2-butylamine at the C-termini yielded the desired mono-TBS ether 4 in high conversion with about 15% and about 23% enantiomeric excess (ee), respectively.

Further optimization of reaction conditions was undertaken with catalyst 6. Additives, such as isopropanol, molecular sieves, etc., either hurt the outcome, or showed no effect on the reaction. Not surprisingly, lowering the temperature resulted in significant improvement in enantioselectivity, but with reduced conversion. When the reaction was run at about −78° C., the desired product 4 was obtained in about 77% ee and about 40% conversion (plus about 2-3% conversion to the bis-silylated product) after about 24 hours. Different silylating agents, like TBSOTf, TBSCN, TBSI were also examined under these reaction conditions. Among these reagents, TBSOTf and TBSI appeared to yield the product with higher conversion (greater than about 60% versus about. 40% with TBSCl), although lower enantioselectivity (about 50-60% ee). On the other hand, TBSCN appeared to be less reactive. Studies into reagent loading suggested about 2.0 equiv TBSCl and about 1.25 equiv DIPEA were optimal, providing the product in about 82% ee with about 40-50% conversion. Finally, the reaction appears to be concentration dependent. A more concentrated reaction (about 0.5 M instead of about 0.2 M) led to about 70% conversion with the same level of enantioselectivity, although more of the bis-silylated side product is also produced (about 12-20%). Conditions which appear to be suitable for this catalyst and substrate combination are summarized in eq 1. These conditions provide the enantioenriched target 4 in about 82% ee; however, additional improvements were thought possible through modifications to the catalyst structure.

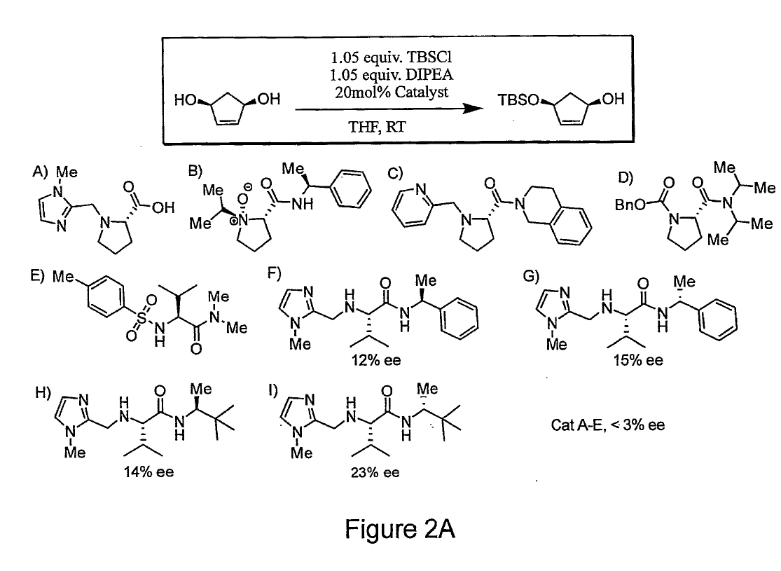

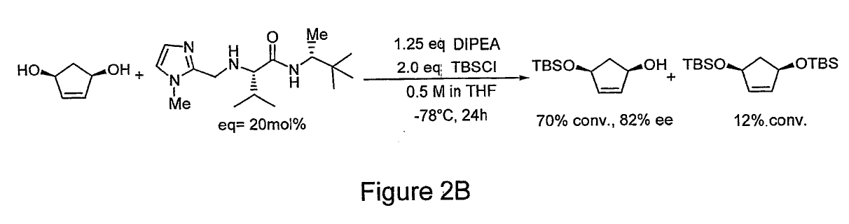

FIG. 2A shows the results of screenings experiments wherein various catalysts were used in the identified reaction. FIG. 2B shows the result of a selected set of conditions in which an about 70% conversion rate was obtained.

By design, the catalysts utilized in these experiments are modular in structure and can be obtained in three steps. Scheme 3 shows the synthesis of catalyst 6 as a representative example; see also FIG. 3. Standard peptide coupling followed by BOC deprotection yields the chiral amine 7, which is transformed to the catalyst via a reductive alkylation step with an appropriate aldehyde 8. The overall yield for the three steps is typically over about 60% and requires only one purification (column chromatography) at the end of the sequence to obtain analytically pure material.

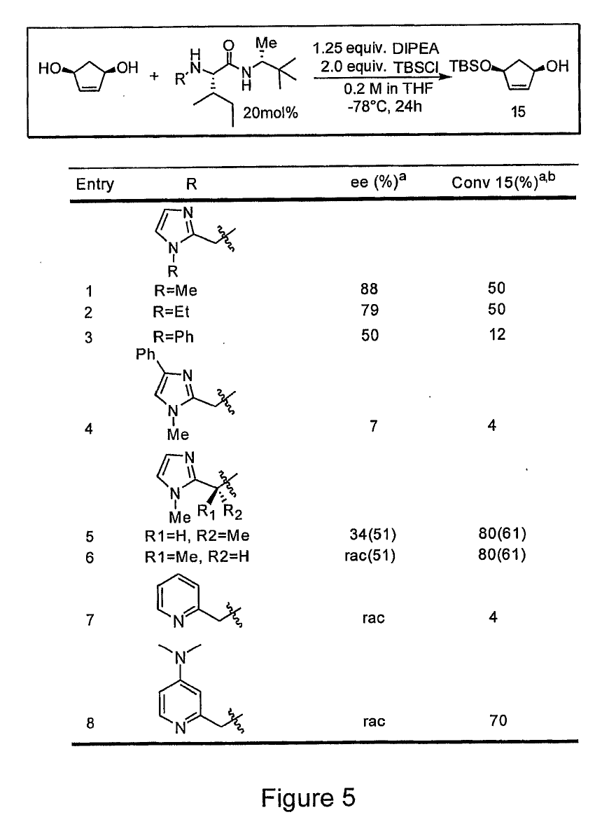

As shown in FIG. 1, the catalyst compound may be divided into three segments: a N-terminal section, an amino acid section, and a C-terminal section. Each section comprises various substituents. Through a positional scanning optimization strategy, the influence of the amino acid, C-termini, and N-termini on catalyst activity in the desymmetrization of substrate 1 at about −78° C. was studied. Certain of these studies are summarized in Scheme 4. Of particular note, the studies indicate that the reaction of this embodiment appears sensitive to the N-terminus identity. Changing the N-methylimidazole moiety appears to hurt the outcome (see also FIG. 5); a thiazolyl moiety was also found to have little or no activity under the screening conditions. (R)-3,3-dimethyl-2-butylamine appeared conducive for the C-terminus. In an experiment, the isoleucine catalyst 9 was more selective than 6, yielding product 4 in about 88% ee and about 50% (0.2 M reaction) conversion.

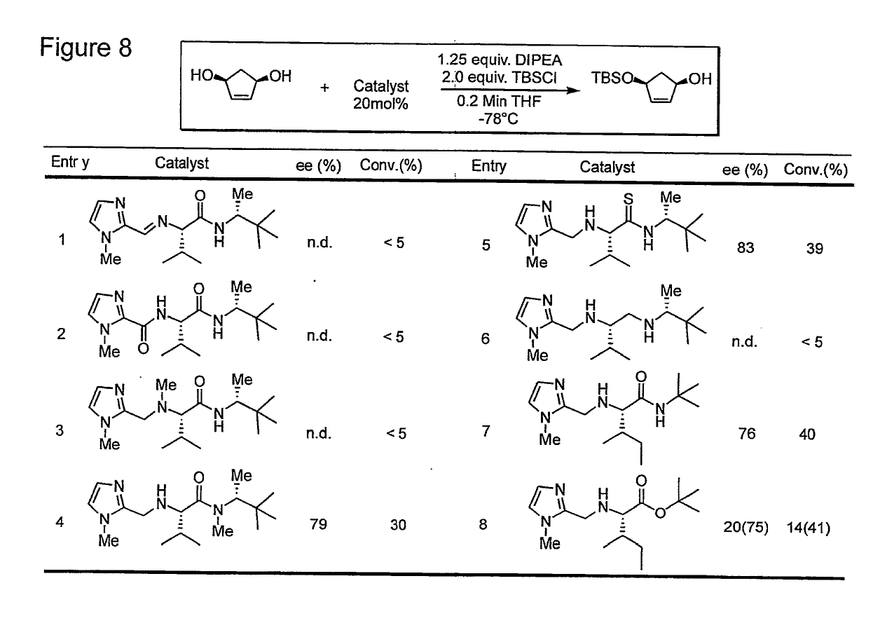

Efforts were made to better understand the structural features of the catalyst that are responsible for the desired activity. These studies are summarized in Table A and FIG. 8. The silaphilic N-methylimidazole moiety, for example, appears relevant for catalytic activity. Changes in this region lead to a decrease in the efficiency of the system (data not shown). Secondly, as illustrated in entries 1-3, the amino acid secondary amine also appears to be a relevant structural feature of the catalyst, since the formation of imine, amide or methylated tertiary amine all destroy the catalytic activity completely. Last, the amide region on the C-terminus of the catalyst is presumably another active site: one possibility is based on the known silaphilicity of amide group, the other is that amide can serve as a hydrogen donor or acceptor, which might interact with the diol substrate to form a highly organized transition state in the reaction. The data in entries 4-8 is consistent with this hypothesis: methylation of the amide nitrogen has no significant effect on the catalytic activity; the thioamide catalyst is also a comparable catalyst for the same reaction, while the reduced catalyst in entry 6 is not active at all. Compared to the tert-butyl amide catalyst in entry 7, the tert-butyl ester catalyst in entry 8 is much less efficient.

TABLE A
Study of catalyst structural features on activity and selectivity.

| | | ee | Conv. |
|---|---|---|---|
| Entry | Catalyst | (%)^{a} | (%)^{a} |
| (1) | | n.d. | <5 |
| (2) | | n.d. | <5 |
| (3) | | n.d. | <5 |
| (4) | | 79 | 30 |
| (5) | | 83 | 39 |
| (6) | | n.d. | <5 |
| (7)^{b} | | 72(75) | 44(41) |
| (8)^{b} | | 20(75) | 14(41) |

^{a}ee determined by Chiral GC analysis.
^{b}The reaction were run at other conditions. Numbers in parentheses are those of reactions catalyzed by 9 under otherwise identical conditions as the reference.

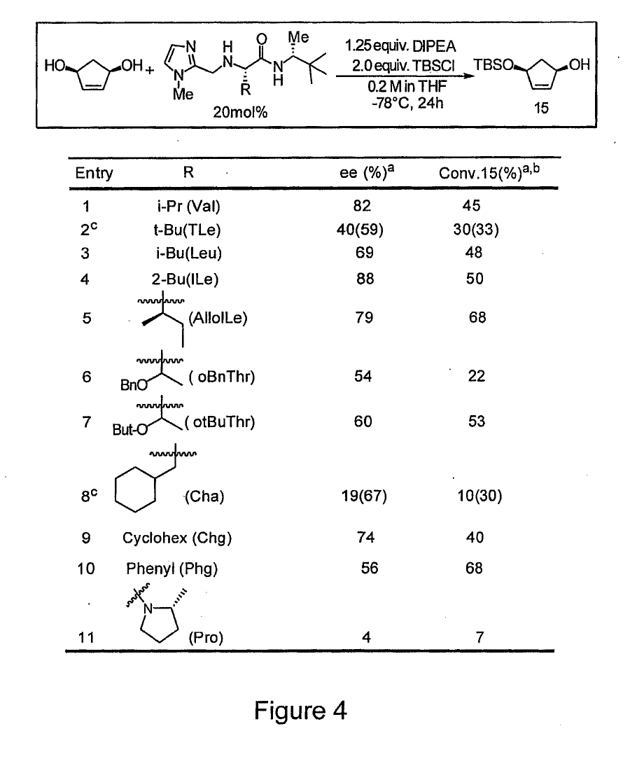

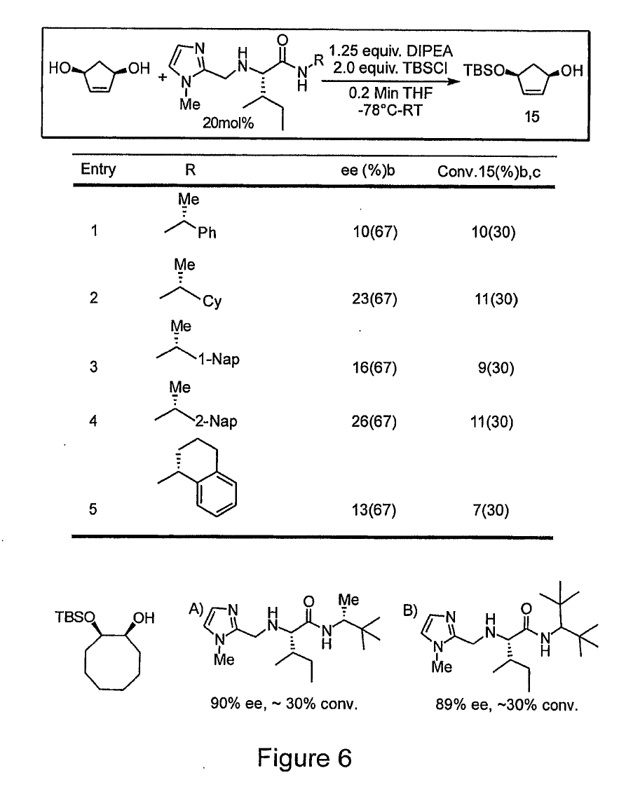

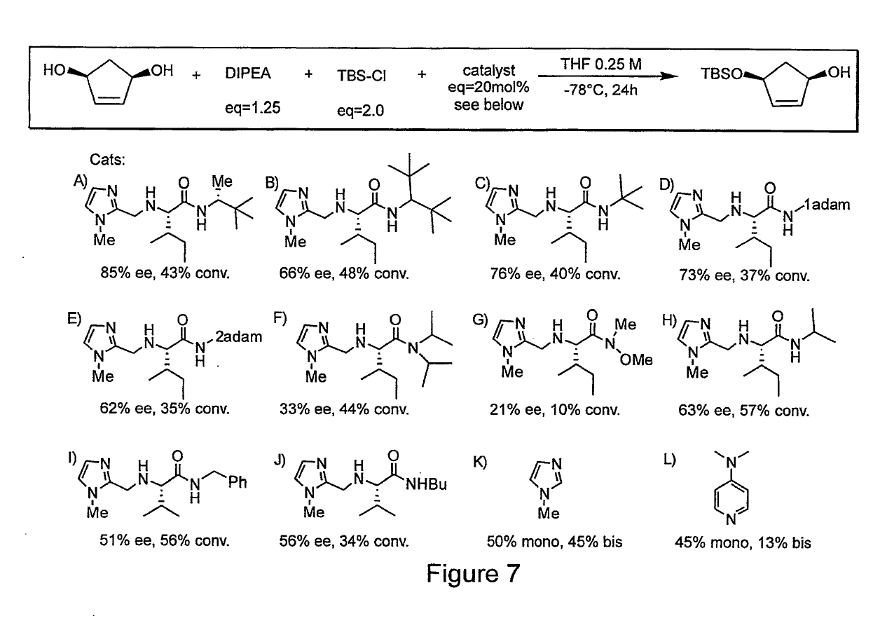

Additional studies of modifications of the catalyst structure are shown in FIGS. 4-7. FIG. 4 shows various amino acid substituents and the effectiveness of the embodiment of the resulting catalyst. FIG. 5 shows various N-termini substituents and the effectiveness of the resulting embodiment of the resulting catalyst. FIG. 6 and FIG. 7 show various C-termini substituents and the effectiveness of the resulting embodiment of the resulting catalyst. With reference to FIG. 7, Catalysts B and C were found to be comparable to Catalyst A for 1,2 diols, while the t-butyl catalyst sidechain proved better for 1,2 diols.

Having identified an efficient catalytic system for the asymmetric silylation of 1,3-diol 1, additional experiments were performed to expand this system to other meso-diols. Keeping in mind the structural differences between the meso-diols, catalysts were tested in combination with various solvents, temperatures, concentrations and reaction times for each substrate. As summarized in Table B, these studies identified effective conditions for a variety of substrates. The reaction shows a generality towards 1,3- and 1,2-diols, either cyclic or acyclic, saturated or unsaturated; all are desymmetrized to yield the mono-TBS ethers in about 88-95% ee's. For the two 1,3-diols tested, the isoleucine-derived catalyst 9 appears selective; for most 1,2-diols, on the other hand, the tert-leucine catalyst 10, appears to be selective. THF and toluene were utilized as solvents for these reactions. Other silyl protective groups, such as TES and TIPS, can also be utilized in this reaction. Examples of these asymmetric silylations were demonstrated with cis-cyclo-octane-1,2-diol. It is also noteworthy for practical purposes that the reactions show a high tolerance towards moisture, air, and reagent impurities. Moreover, the catalyst can be recovered about >95% and reused without any noticeable decrease in reactivity or selectivity after the reaction is complete through a simple aqueous acid extraction protocol.

TABLE B
Desymmetrization of meso-diols. ^{a,b}

| | |
|---|---|
| | |
| | |
| | |
| | |
| | |
| | |

^{a} ee determined by chiral GC analysis.
^{b} yields represent isolated yield based on at least two runs.
^{c} volatility issues and over silylation products appeared to lower the isolated yield of 4 relative to other substrates.

Certain other substrates were also tested, as shown below:

In conclusion, the first desymmetrization of meso-diols via direct asymmetric silylation has been achieved. This system shows high generality towards a variety of 1,2- and 1,3-diols and in all cases, good to excellent enantioselectivities are obtained. The catalyst is amino acid derived, modular and easily synthesized in three steps from inexpensive, commercially available materials. This asymmetric transformation represents a convenient and powerful new tool in organic synthesis.

FIG. 8 shows data relating to the use of various embodiments of the presently disclosed catalysts in a particular reactive system. As discussed in the experimental section above, the data provides a mechanistic insight as to the active sites of the illustrated catalysts.

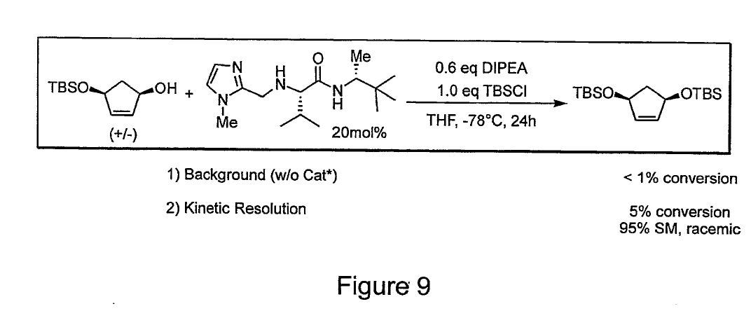

FIG. 9 shows a kinetic resolution test of certain catalysts. The results indicate the participation of both hydroxy groups of the substrate in the reaction. In addition, the results indicate a dual activation by the catalyst (silaphilic activation of TBSCl; hydrogen bond with diol to form highly organized transition state which dictates the enantioselectivity of the reaction.)

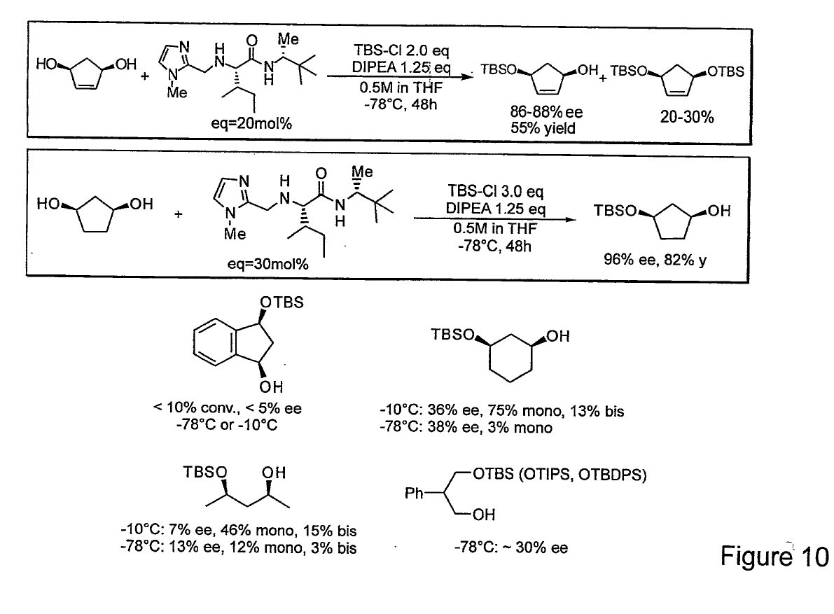

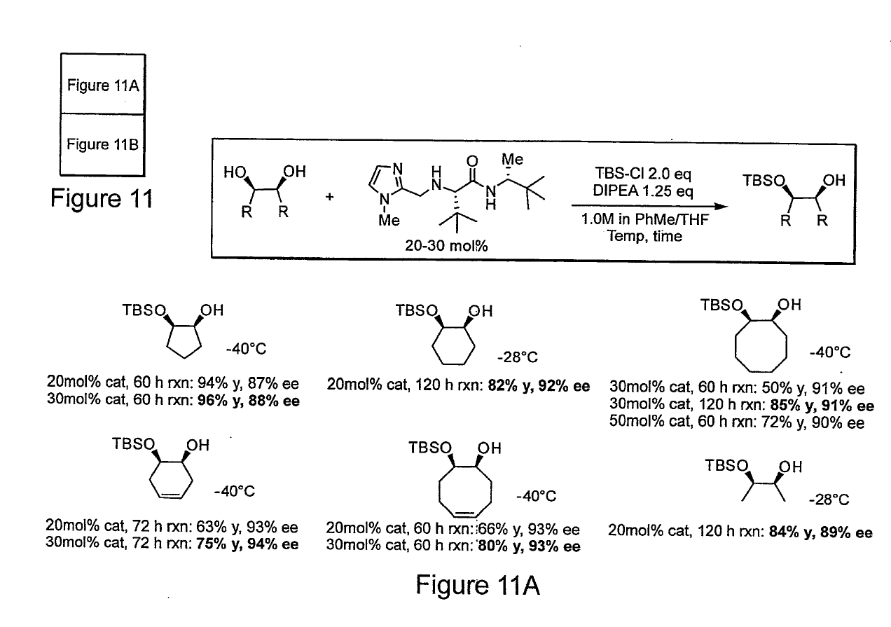

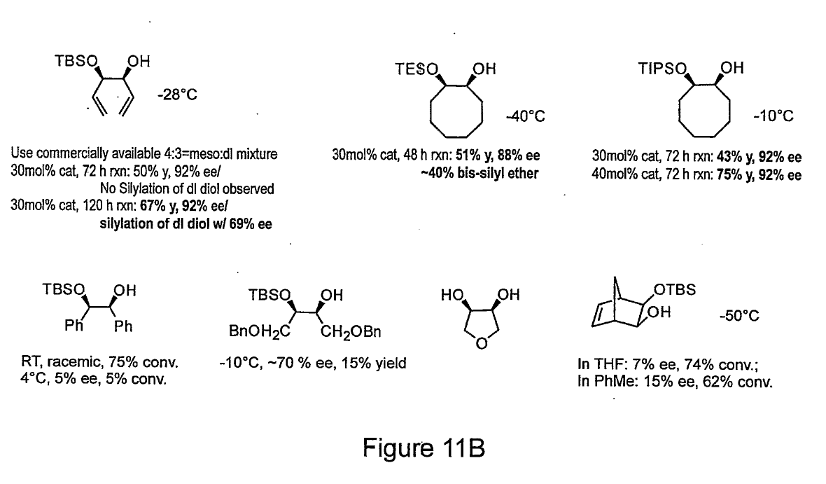

FIGS. 10-11 show the effect of a catalyst of the invention on various types of substrate. FIG. 10 shows the effect of an embodiment of a catalyst on various 1,3-diols. FIG. 11 shows the effect of reaction in the presence of a catalyst of the invention on various 1,2-diols. Those skilled in the art will recognize that various substrates are within the spirit and scope of the present invention.

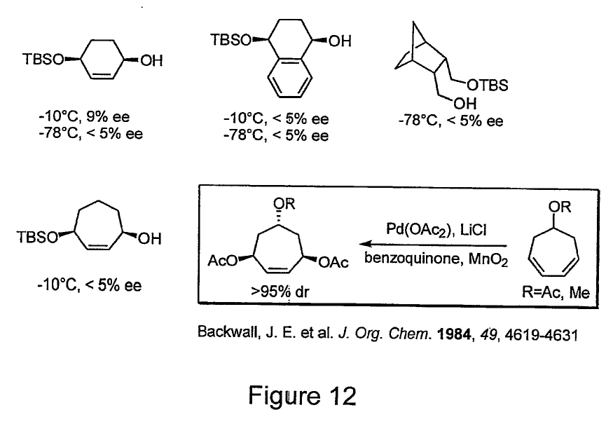

FIG. 12 shows the use of a catalyst of the invention to produce kinetic resolution of diols. As shown, presently disclosed selective silylation can be used to differentiate between and thereby separate racemic diols.

### Example 4

Reliable, effective and environmentally friendly chemical transformations are crucial to the ability of scientists to study biological events, develop new therapeutics and design novel materials. Discovery of chiral catalysts that can be easily prepared and provide access to organic molecules of high optical purity is a critical goal of modern chemical synthesis. On a related front, the development of protecting groups that shield a molecule's functionality from undergoing an undesired transformation has proven to be indispensable in the preparation of many complex biologically active molecules. Chiral catalysts that install a protecting group and, simultaneously, convert an achiral molecule to one that is chiral and optically enriched, can thus increase the efficiency of many synthetic pathways. Here a new chiral catalyst is presently disclosed that promotes the enantioselective shielding of a secondary alcohol as what is probably its most commonly used protected form, a silyl ether. In this experiment, the catalyst is a small-molecule peptide prepared in three steps from commercially available materials. Enantioselective silylations are performed in the presence of commercially available silyl chlorides and afford products in up to about 96% enantiomeric excess (e.e.) and in yields of up to 96%.

The availability of a chiral catalyst for silylation of alcohols can have a significant impact on the efficiency with which optically enriched organic molecules are prepared. The example provided below illustrates this point. Unsaturated silyl-protected ketone 4 is a valuable building block used in the synthesis of several biologically active entities in the optically active form (e.g., neocarzinostatins, prostoglandins, prostacycline, thromboxane and various nucleosides). Not surprisingly, a considerable range of procedures has been disclosed for the preparation of optically enriched 4. Perhaps the most efficient route, in terms of overall yield, amount of waste produced and the time required, is the eight-step procedure shown below (via 2 and 3). This sequence was developed to avoid another protocol that involves three chemical transformations but demands an enzymatic (germ lipase) de-acylation that consumes ten days: seven days for the enantioselective reaction, followed by a three day procedure for isolation of the desired product that also generates copious amounts of solvent waste. A chiral catalyst that directly converts achiral diol 1 to optically enriched silyl ether 3 by a catalytic enantioselective silylation would present a direct two-step sequence to optically enriched 4. A chiral acylation catalyst would reduce the sequence below by only one step.

As shown above, a catalyst for enantioselective silylation of alcohols can render synthesis of an important organic molecule such as cyclopentenone 4 significantly more efficient.

To initiate the current search for a chiral silylation catalyst, two design criteria were considered. One requirement was that the catalyst contains a nucleophilic moiety for interaction with and activation of the silyl halide reagent. The other criterion was the presence of H-bonding sites that promote catalyst-substrate association, allowing the catalyst to serve as a platform that brings together the substrate and the activated silylating agent. Guided by the above principles, and with the conversion of 1 to 3 in the presence of commercially available TBSCl (tert-butyldimethylsilyl chloride) serving as the representative process, the ability of several molecules were evaluated as potential chiral silylation catalysts. The molecules selected as catalyst candidates carry amino acid units (H-bonding sites) and fictional groups expected to readily react with silyl chlorides (e.g., N-oxide 5, N-alkylimidazoles 6-7, and pyridine 8). It was established that, in contrast to compounds such as 5, 7 and 8 (see Table 1), which only generate racemic 3 (50:50 enantiomeric ratio or e.r.), silylation of 1 in the presence of 6 affords 3 with measurable optical enrichment (58:42 e.r., 16% e.e.). This observation was encouraging, since control experiments indicated that uncatalyzed reaction (affording racemic product) can also occur to a small extent under the reaction conditions (at 22° C.). These findings suggested that higher enantioselection could perhaps be induced by the catalyzed process than the data in entry 2 of Table 1 indicate. When the silylation was performed at about −78° C. (to minimize uncatalyzed reaction), silyl ether 3 was isolated with significantly improved optical purity (91:9 e.r. or 82% e.e.). Reactions at about −78° C. but in the presence of 5-7 afforded racemic 3.

TABLE 1

| Entry no | Catalyst | Equiv.* | e.r.** | e.e.*** (%) |
|---|---|---|---|---|
| 1 | 5 | 0.2 | 50:50 | <5 |
| 2 | 6 | 0.2 | 58:42 | 16 |
| 3 | 7 | 0.2 | 50:50 | <5 |
| 4 | 8 | 0.2 | 50:50 | <5 |

*The reactions were carrier out in purified THF in the presence of 1.25 equiv. of DIPEA at 22° C. for 20-24 hours and with a substrate concentration of about 0.2 M.
**The enantiomeric ratio (e.r.) was determined by chiral gas liquid chromatography (GLC) analysis.
***The enantiomeric excess (e.e.) was calculated from the e.r.; the variances of e.e. are estimated to be about <2%.

As shown, Table 1 illustrates initial screening of catalyst candidates for enantioselective silylation of substrate 1 (to obtain 3).

At this point, the facile modularity of peptidic structures was exploited and analogues of 6 were easily and expeditiously prepared and their catalytic activities examined. These studies indicated that in the presence of about 0.3 equivalents of peptide 9 and two equivalents of TBSCl, enantioselective silylation of 1 leads to the formation of 3 (see entry 1, Table 2, below) in 91.5:9.5 e.r. (about 81% e.e.) and about 54% isolated yield (>98% conversion to product in about 24 h). Further studies, the results of which are summarized in Table 2, established that 9 promotes the enantioselective silylation of a range of achiral diols, giving rise to the desired chiral silyl ethers in excellent optical purity (≧94:6 e.r. or ≧88% e.e.). Table 2 shows enantioselective silylations of achiral diols catalyzed by catalyst 9.

TABLE 2
Enantioselective silylations of achiral diols catalyzed by catalyst 9

| | |
|---|---|
| | |

| Entry no | Product | Catalyst Equiv.* | Time (h) | Temp (° C.) | o.r. † | e.e.‡ (%) | Yield§ (%) |
|---|---|---|---|---|---|---|---|
| 1 | | 0.3 | 24 | −78 | 97.5:2.5 | 81 | 54 |
| 2 | | 0.2 | 24 | −78 | 98:2 | 96 | 82 |
| 3 | | 0.3 | 60 | −40 | 94:6 | 88 | 96 |
| 4 | | 0.2 | 120 | −28 | 96:4 | 92 | 82 |
| 5 | | 0.3 | 72 | −50 | 97:3 | 94 | 75 |
| 6 | | 0.3 | 72 | −40 | 96.5:3.5 | 93 | 93 |
| 7 | | 0.3 | 120 | −40 | 95.5:4.5 | 95 | 96 |
| 8 | | 0.3 | 72 | −50 | 96.5:3.5 | 93 | 80 |
| 9 | | 0.2 | 120 | −28 | 94.5:5.5 | 89 | 84 |
| 10 | | 0.3 | 72 | −28 | 96:4 | 92 | 67 |

*The reactions were carried out in purified THF or toluene in the presence of TBSCl, 1.25 equiv of DIPEA, and with a substrate concentration of 0.5 or 1.0 M (see the Supplementary information for details).
† The enantiomeric raio (e.r.) was determined by chiral gas liquid chromatography (GLC) analysis. See the Supplementary information for details.
‡ The enantiomeric excess (e.e.) was calculated from the e.r.; the variance of e.e. are estimated to be <±2%.
§ Yield of isolated product after purification (see in the Supplementary information for details).

Several points regarding the catalytic enantioselective silylations are noteworthy. First, catalyst 9, a small peptide (molecular weight=308.5 g/mole), is prepared from commercially available materials in about 60-70% overall yield by a straightforward three-step sequence. This catalyst preparation can deliver analytically pure catalyst after only one silica gel chromatography. After reaction, the chiral catalyst can often be recovered, along with the desired product, by silica gel chromatography (near quantitative yield) and re-used with the same levels of efficiency and enantioselectivity as observed initially. Second, as the results summarized in entries 3-10 of Table 2 indicate, catalytic silylations can be performed on 1,2-diols (in addition to 1,3-diols) with high enantioselectivity. Catalytic enantioselective silylations are not limited to reactions of five-membered rings: cyclohexanediols (entries 4-5) and medium-sized ring substrates (entries 6-8) are transformed to silyl ethers with about >92% e.e. (about >96:4 e.r.). As the examples in entries 9-10 of Table 2 indicate, acyclic substrates can be desymmetrized by enantioselective silylation to afford products of high optical purity (about >88% e.e. or >94:6 e.r.) and in yields that are useful in chemical synthesis.

Third, catalytic asymmetric silylations are not limited to reactions with TBSCl. As the representative examples below illustrate, other commonly used silyl chlorides, such as TESC1 (triethylsilyl chloride) and TIPS [tri-(iso-propyl)silyl chloride] can be employed to afford the desired optically enriched compounds. Fourth, although in some reactions small amounts (about <10%) of bis-silylated products are isolated, high enantioselectivities are not due to rapid second silylation of the minor product enantiomer (kinetic resolution). This was established through subjection of racemic products to the reaction conditions. After the appropriate time (see Table 2 for the required times), about <2% bis-silylated compound was observed, and the enantiomeric excess (e.e.) of the silyl ether remained the same. As shown below, enantioselective silylations of achiral diols with different silyl chloride reagents catalyzed by catalyst 9. TES=triethylsilyl; TIPS=tri(iso-propyl)silyl:

Without wishing to be bound by any particular theory, it is believed that the transition state model depicted below may account for the enantioselective silylations catalyzed by 9. Substrate-catalyst association by H-bonding between the hydroxyl groups and the peptidic side chain results in preferential silylation of one enantiotopic alcohol. Based on principles initially put forth by Guttmann, the chiral catalyst's heterocyclic moiety can enhance the reactivity of the silylating agent towards nucleophilic attack. Thus, by serving as a donor ligand to the Si center, the imidazole moiety promotes re-distribution of electron density and enhancement of Si electrophilicity (due to increased build-up of electron density at silicon's ligands). The suggested scenario is supported by the significant change in catalytic activity exhibited by various related small peptides that are structurally modified (see Table 3). Thus, compound 10, bearing a less nucleophilic thiazole group is ineffective as a catalyst (entry 1, Table 3). Schiff base 11 (entry 2, Table 3) is inactive; the N atom of the C═N in 11 (vs C—N in 9) may not be able to H-bond effectively with the substrate hydroxyl group (see FIG. 3) due to geometric constraints. Amide 12 (entry 3, Table 3) is inactive, likely because the H-bond acceptor has been removed from the catalyst structure. The Lewis basic but sterically more hindered tertiary amine 13 is slightly less active (about 30% conversion after about 24 h at about −78° C. vs. about 40% conversion with 9) and affords 3 with lower enantioselectivity (about 79% e.e. vs. about 81% e.e. with 9); H-bonding in the manner illustrated below would destabilize the proposed mode of reaction as it would engender unfavorable steric interactions between the amine methyl and the large tert-butyl group of the tert-Leu moiety. The importance of the H-bonding between the amide carbonyl group and the alcohol group that remains unprotected is underlined by complete loss of activity caused by the removal of the amide carbonyl of the tert-Leu moiety (14). Thus, the less Lewis basic thiocarbonyl of 15 offers appreciable (about 83% e.e. vs about 95% e.e. with 9) enantioselectivity but at a reduced rate (about 40% conversion vs. about >98% conversion with 9), and the still lower Lewis basicity of tert-butyl ester of 16 translates to an even less efficient (about 14% conversion) and enantioselective (about 20% e.e.) outcome. A study of enantioselective silylations with samples of 9 that are of varying levels of optical purity clearly indicates that there is a strictly linear relationship between catalyst and product e.e. (see, e.g, the Supplementary Information in Y. Zhao et al., Nature (2006) 443(7107):67-70, incorporated herein by reference); this finding suggests that complexation and catalysis by complexes that consist of several molecules of 9 is unlikely. Table 3 below shows the effect of variations in catalyst structure on enantioselective synthesis of 3.

TABLE 3
Effect of variations in catalyst structure on enantioselective synthesis of 3

| Entry no | Catalyst | Conv.* (%) | e.r.† | e.e.‡ (%) |
|---|---|---|---|---|
| 1 | 10 | <5 | — | — |
| 2 | 11 | <5 | — | — |
| 3 | 12 | <5 | — | — |
| 4 | 13 | 30 | 89.5:10.5 | 79 |
| 5 | 14 | <5 | — | — |
| 6 | 15 | 40 | 91.5:8.5 | 83 |
| 7 | 16 | 14 | 60:40 | 20 |

*The reactions were carried out with 0.2 equiv. of catalyst and 1.25 equiv DIPEA in purified THF at −78° C. for 24 hours.
Conversion determined by analysis of 400 MHz ¹H NMR spectra of unpuried product mixtures.
† The enantiomeric ratio (e.r.) was determined by chiral gas liquid chromatography (GLC) analysis. See the Supplementary information for details.
‡ The enantiomeric excess (e.e.) was calculated from the e.r.; the variance of e.e. are estimated to be <±2%.

As discussed above, and without being bound by theory, the below figure illustrates a proposed transition state model for enantioselective formation of 3:

In conclusion, it is herein disclosed the first example of a chiral silylation catalyst that induces high optical purity while masking an alcohol by one of the most widely used protecting groups in organic chemistry. In addition to pointing to the need for more efficient catalysts (lower catalyst loading and shorter reaction times), the present investigations raise several important and interesting questions. Among these is the possibility of using the chiral silylation catalyst to modify structurally, with high site selectivity, an optically enriched molecule that contains numerous alcohol functionalities (see infra).

### Example 5

### Kinetic Resolution of Racemic Unsymmetrical Cis-Diols

The catalysts of the present invention are also useful for the kinetic resolution of unsymmetrical polyhydroxyl-containing compounds. Several examples of this transformation are provided in Scheme 5. In these cases, the less hindered alcohol for only one enantiomer is selectively silylated using our peptide-based imidizole catalyst. This allows us to isolate the unreacted diol with high enantiomeric purity.

As illustrated in Scheme 6, this kinetic resolution strategy was shown to be amenable to systems containing primary hydroxyl groups as well. In these examples, the less hindered primary alcohol is selectively protected for only one enantiomer. In some cases, this allows for both the mono-silylated product and starting diol to be isolated with high enantiomeric purity.

For poly-hydroxyl-containing systems with less steric differentiation, we have been pursuing a parallel kinetic resolution strategy. In this case, we typically observed two silylated products, where each mono-silylated regioisomer is enantiomerically enriched. These results are summarized in Scheme 7.

### Example 6

### Elaboration of Silylated Products

With the goal of addressing other asymmetric challenges, such as allylations, we have shown that allylated silyl groups can be also introduced in meso-diols with high selectivity using our catalytic system. These results are summarized in Scheme 4. We are currently using these enantiomerically enriched allyl silanes in various asymmetric allylation processes, also shown in Scheme 8.

### Example 7

### Resolution of β-hydroxyketones

The amino acid-based catalysts of the invention can also be used to resolve β-hydroxyketones. Table 4 summarizes our findings along these lines. For the reaction illustrated in entry 1 of Table 4, we have shown the catalyst loading can go as low as 0.05 mol % without loss of enantiomeric excess (selectivity) or conversion (reactivity). Catalyst loading of 0.01 mol % still provided product (20% conversion, 18% ee(sm), 71% ee(prod)). The present catalysts can also be used for dynamic kinetic resolution.

TABLE 4
Kinetic resolution of β-hydroxyketones.

| Entry | Substrate | Conversion [%] | ee(sm) [%] | ee(prod) [%] | Yield (prod) [%] | krel |
|---|---|---|---|---|---|---|
| 1 | | 56 | 84 | 67 | | 14 |
| 2 | | 57 | 88 | 67 | | 15 |
| 3 | | 55 | 82 | 67 | | 13 |
| 4 | | 57 | 79 | 60 | | 9 |
| 5 | | 60 | 81 | 55 | | 8 |
| 6 | | 75 | 91 | 31 | | 6 |
| 7 | | 74 | 92 | 32 | | 5 |
| 8 | | 63 | 12 | 7 | | 1 |

All patents, patent applications, and published references cited herein are hereby incorporated by reference in their entirety. It will be appreciated that various of the above-disclosed and other features and functions, or alternatives thereof, may be desirably combined into many other different systems or applications. Various alternatives, modifications, variations, or improvements therein may be subsequently made by those skilled in the art which are also intended to be encompassed by the following claims.

1. A method for direct asymmetric silylation of an alcohol, comprising:
reacting the alcohol with a silylating agent in the presence of a chiral catalyst, to produce an enantiomerically enriched silylated product.

2. The method of claim 1, wherein the catalyst is represented by the formula:
wherein:
R₁ is H, C₁-C₆ alkyl, aryl, or heteroaryl;
R₂ is, independently for each occurrence, C₁-C₆ alkyl, aryl, or heteroaryl;
Y is O, S or N—R₃;
Z is, independently for each occurrence, O or S;
R₃ is H, C₁-C₆ alkyl, aryl, or heteroaryl;
R₄ is (CH₂)ₘ—X;
X is aryl or heteroaryl;
m is 0, 1 or 2; and
n is an integer from 1 to 10;
or a salt thereof.

3. The method of claim 2, wherein Y is N—R₃.

4. The method of claim 2, wherein Z is O for each occurrence.

5. The method of claim 1, wherein the catalyst is represented by the formula:
wherein
R₁ is H, C₁-C₆ alkyl, aryl, or heteroaryl;
R₂ is C₁-C₆ alkyl, aryl, or heteroaryl;
R₃ is H, C₁-C₆ alkyl, aryl, or heteroaryl;
R₄ is (CH₂)ₘ—X;
X is aryl or heteroaryl;
m is 0, 1 or 2; and
n is an integer from 1 to 10;
or a salt thereof.

6. The method of claim 5, wherein X is heteroaryl.

7. The method of claim 6, wherein X is a nitrogen-containing 5- or 6-membered heteroaryl group.

8. The method of claim 7, wherein X is a 1-methylimidazol-2-yl group.

9. The method of claim 1, wherein the catalyst can be represented by the formula:
in which:
R₂ is a straight or branched alkyl group;
R₅ is an aryl or alkyl group;
R₆ is a lower alkyl;
R₇ is a lower alkyl; and
n=1, 2, 3, 4, 5, 6, 7, 8, 9, or 10;
or a salt thereof.

10. The method of claim 1, wherein the catalyst is represented by the formula:
wherein:
R₂ is a straight or branched alkyl group;
R₅ is:
R₆ is a lower alkyl; and
R₇ is a lower alkyl;
n=1, 2, 3, 4, 5, 6, 7, 8, 9, or 10.

11-16. (canceled)

17. The method of claim 1 wherein the alcohol is a diol.

18. The method of claim 1 wherein the alcohol is a 1,2 cyclic diol.

19. A compound represented by the formula:
wherein:
R₂ is a straight or branched alkyl group;
R₅ is an aryl or alkyl group;
R₆ is a lower alkyl;
R₇ is a lower alkyl; and
n=1, 2, 3, 4, 5, 6, 7, 8, 9, or 10;
or a salt thereof.

20-25. (canceled)

26. The compound of claim 19, wherein the compound is represented by the formula:
wherein:
R₅ is phenyl or t-butyl; and
R₈ and R₉ are each independently H, alkyl or aryl;
or a salt thereof.

27. The compound of claim 26, wherein the compound is represented by the formula:

28. The compound of claim 26, wherein R₅ is t-butyl.

29. The compound of claim 26, wherein R₅ is phenyl.

30. The compound of claim 26, wherein R₈ and R₉ are each independently selected from methyl and ethyl.

31. (canceled)

32. A method of silylating a hydroxy group of a compound comprising at least one hydroxy group, the method comprising:
contacting the compound with a catalyst of the formula:
in which:
R₁ is H, C₁-C₆ alkyl, aryl, or heteroaryl;
R₂ is, independently for each occurrence, C₁-C₆ alkyl, aryl, or heteroaryl;
Y is O, S or N—R₃;
Z is, independently for each occurrence, O or S;
R₃ is H, C₁-C₆ alkyl, aryl, or heteroaryl;
R₄ is (CH₂)ₘ—X;
X is aryl or heteroaryl;
m is 0, 1 or 2; and
n is an integer from 1 to 10;
or a salt thereof;
in the presence of a silylating reagent and a base;
under conditions such that the hydroxy group of the compound is silylated.

33. The method of claim 32, wherein the compound is a diol.

34. The compound of claim 33, wherein the diol is a 1,2-diol.

35. (canceled)

36. The method of claim 32, wherein the compound is a beta-hydroxy ketone.

37. The method of claim 32, wherein the base is an organic base.

38. The method of claim 32, wherein the catalyst is represented by the formula:
wherein:
R₂ is a straight or branched alkyl group;
R₅ is an aryl or alkyl group;
R₆ is a lower alkyl;
R₇ is a lower alkyl; and
n=1, 2, 3, 4, 5, 6, 7, 8, 9, or 10;
or a salt thereof.

39. The method of claim 32, wherein the catalyst is represented by the formula:
wherein:
R₅ is phenyl or t-butyl; and
R₈ and R₉ are each independently H, alkyl or aryl;
or a salt thereof.

40. (canceled)

41. (canceled)

42. A method of kinetic resolution of a mixture of two stereoisomers of an unsymmetrical diol, the method comprising:
contacting the mixture of two stereoisomers of the unsymmetrical diol with a catalyst of the formula:
in which:
R₁ is H, C₁-C₆ alkyl, aryl, or heteroaryl;
R₂ is, independently for each occurrence, C₁-C₆ alkyl, aryl, or heteroaryl;
Y is O, S or N—R₃;
Z is, independently for each occurrence, O or S;
R₃ is H, C₁-C₆ alkyl, aryl, or heteroaryl;
R₄ is (CH₂)ₘ—X;
X is aryl or heteroaryl;
m is 0, 1 or 2; and
n is an integer from 1 to 10;
or a salt thereof;
in the presence of a silylating reagent and a base;
under conditions such that a hydroxy group of one of the stereoisomers of the unsymmetrical diol is selectively silylated compared to another stereoisomer of the unsymmetrical diol.

43. The compound of claim 42, wherein the unsymmetrical diol is a 1,2-diol.

44. The method of claim 42, wherein the unsymmetrical diol is a triol.

45. The method of claim 42, wherein the base is an organic base.

46. The method of claim 42, wherein the catalyst is represented by the formula:
wherein:
R₂ is a straight or branched alkyl group;
R₅ is an aryl or alkyl group;
R₆ is a lower alkyl;
R₇ is a lower alkyl; and
n=1, 2, 3, 4, 5, 6, 7, 8, 9, or 10;
or a salt thereof.

47-49. (canceled)

50. A reaction mixture comprising:
(i) a catalyst of the formula:
in which:
R₁ is H, C₁-C₆ alkyl, aryl, or heteroaryl;
R₂ is, independently for each occurrence, C₁-C₆ alkyl, aryl, or heteroaryl;
Y is O, S or N—R₃;
Z is, independently for each occurrence, O or S;
R₃ is H, C₁-C₆ alkyl, aryl, or heteroaryl;
R₄ is (CH₂)ₘ—X;
X is aryl or heteroaryl;
m is 0, 1 or 2; and
n is an integer from 1 to 10;
or a salt thereof;
(ii) a silylating reagent; and
(iii) a base.

51. The reaction mixture of claim 50, further comprising a solvent.

52. The reaction mixture of claim 50, further comprising a 1,2-diol.

53. The reaction mixture of claim 50, wherein the base is an organic base.
